(19) United States
(12) Patent Application Publication
(10) Pub. No.: US 2002/0010401 A1
(43) Pub. Date: Jan. 24, 2002

(54) Pre- and post-processing of spectral data for calibration using mutivariate analysis techniques

(76) Inventors: Andrew Bushmakin, Nashua, NH (US); James R. Mansfield, Boston, MA (US); Pierre Trepagnier, Medford, MA (US)

Correspondence Address: HELLER EHRMAN WHITE & MCAULIFFE LLP, SUITE 300, 101 ORCHARD RIDGE DR., GAITHERSBURG, MD, 20878-1917, US

(21) Appl. No.: 09/855,755

(22) Filed: May 16, 2001

Related U.S. Application Data
(60) Provisional application No. 60/205,103, filed on May 18, 2000.

(51) Int. Cl. A61B 6/00

(52) U.S. Cl. 600/476; 600/477; 600/310; 356/39

(57) ABSTRACT

This invention relates to a method for quantitating the relationship between an analyte level in in vivo tissue and the auto-fluorescent spectral characteristics in the tissue.

### RELATED APPLICATION

The present invention claims priority to U.S. Provisional Application No. 60/205,103, filed on May 18, 2000.

### BACKGROUND OF THE INVENTION

1. Field of the Invention

This invention relates to the processing of in-vivo tissue native auto-fluorescence spectra for the purposes of non-invasively determining blood glucose levels.

2. Description of the Background

Changes in skin fluorescence spectra due to changes in blood glucose levels have been observed. See U.S. patent application Ser. No. 09/785,547, titled “Non-Invasive Tissue Glucose Level Monitoring,” filed Feb. 18, 2001, which is a continuation-in-part of U.S. patent application Ser. No. 09/287,486, titled “Non-Invasive Tissue Glucose Level Monitoring,” filed Apr. 6, 1999; both incorporated herein by reference.

Peak ratios, correlation analysis, and linear regression analysis have been used to analyze skin autofluorescence spectra for the purpose of determining the blood glucose concentration. Although correlations have been shown, these have not been sufficient for quantitation of blood glucose levels.

When faced with complex calibration requirements for analytical methods, it is common to apply multivariate statistical methods to the analysis. Multivariate statistical methods have long been used in the analysis of biomedical samples by infrared and near infrared, generally under the name “chemometrics.” See, e.g., U.S. Pat. No. 5,596,992 to Haaland et al. and U.S. Pat. No. 5,857,462 to Thomas et al. The most common multivariate calibration methodology employed in the field of spectroscopy is partial least squares (“PLS”).

There are also many agricultural applications of near-IR spectroscopy and PLS processing. Near-IR spectra taken from agricultural samples (such as, grains, oil seeds, feeds, etc.) have been used to quantitate various bulk constituents (such as, total protein, water content, fat content, etc.).

The use of multivariate methods for the analysis of ex-vivo tissue samples is well established. For spectra taken in-vivo, there has been some work done. Linear discriminant analysis has been used to classify visible/near-IR spectra of human finger joints into early and late rheumatoid arthritis classes. PLS analysis of near-IR spectra is the basis of all infrared efforts towards non-invasive glucose monitoring. Multivariate methods have been used to classify fluorescence spectra taken in-vivo from cervixes according to the presence or absence of cervical cancer or pre-cancerous tissues.

In general, the field of chemometrics is well established, and the use of multivariate statistical methods for the analysis of complex spectra is common. These methods are used in pharmaceutical analysis, industrial applications, and, more recently, biomedical spectral analysis.

Standard chemometric techniques have been applied to the analysis of tissue autofluorescence spectra for the purpose of non-invasively quantitating in vivo levels of blood glucose, with only marginal success. Methods such as linear regression, multiple linear regression and stepwise linear regression are not able to create a calibration for blood glucose levels with tissue autofluorescence spectra. Partial least squares methods on their own, even in combination with standard spectroscopic preprocessing methods such as smoothing, derivatives, area and peak normalization or peak enhancement/deconvolution, have some utility, but are clearly insufficient for the task of developing a commercial non-invasive blood glucose analyzer based on tissue autofluorescence techniques.

PLS calibration models created using tissue autofluorescence spectra and glucose values processed using standard spectroscopic methods show only a small tendency for their predicted glucose values to trend with actual glucose values. Standard spectral pre-processing methods include smoothing, derivatives, peak normalization, area normalization, mean centering and variance scaling. Glucose values were processed by use of standard mean centering and variance scaling techniques. In addition to using the standard mean centering and variance scaling on calibration data sets as a whole, they were also used on a per-subject basis within multi-subject data sets, with little success.

### SUMMARY OF THE INVENTION

The present invention overcomes the problems and disadvantages of current strategies and designs, and provides a method for quantitating the relationship between an analyte level in in vivo tissue and the auto-fluorescent spectral characteristics in the tissue. One such method comprises generating a single excitation wavelength or plurality of different excitation wavelengths of green to ultraviolet light; irradiating the tissue with the light and measuring the intensity of the stimulated emission of the sample at a minimum of two different wavelengths of lower energy than the excitation light or at a plurality of wavelengths of lower energy than the excitation light; applying a transformation to the wavelength data; analyzing the transformed data; and inverting the original transformation to yield analytical results in standard units.

Preferably, the analyte is glucose and the tissue is skin. Preferably, the relative transformations of glucose and spectra are selected from the group comprising or, alternately, consisting of, the single-point transformations (g|s)ₖ=(G|S)ₖ−(G|S)_{N} or (g|s)ₖ=(G|S)ₖ÷(G|S)_{N} and the point-by-point transformations (g|s)ₖ=(G|S)ₖ−(G|S)ₖ₋₁ or (g|s)ₖ(G|S)ₖ÷(G|S)ₖ₋₁.

Another embodiment is directed to a method of quantitating the relationship between an analyte level in tissue and the absorption spectrum of the tissue, wherein the concentration of the analyte is not being directly measured, but rather indirectly inferred through its effect on components of the tissue. This method comprises: irradiating the tissue with electromagnetic radiation and measuring the absorption spectrum of the electromagnetic radiation; applying a relative transformation to the spectral data and another relative transformation to the analyte, the relative transformation in each case being selected from a group comprising either point-by-point or single-point relative transformations; analyzing the transformed data using multivariate techniques; and inverting the original transformation to yield analytical results in standard units.

In one embodiment, the electromagnetic radiation is near-ultraviolet to visible light. Alternately, the electromagnetic radiation may be visible to near-infrared light. In still another embodiment, the electromagnetic radiation is infrared radiation.

Other embodiments and advantages of the invention are set forth in part in the description which follows, and in part, will be obvious from this description, or may be learned from the practice of the invention.

### DESCRIPTION OF THE DRAWINGS

FIG. 1 is a flow diagram for glucose calibration.

### DESCRIPTION OF THE INVENTION

Creating a marketable product for the non-invasive monitoring of glucose using fluorescence excitation spectroscopy requires the analysis of large numbers of spectra from a large population of individuals, and the creation of algorithms which convert spectral data from this population into glucose values. A single algorithm may work for everybody, or the large populations may well separate into a relatively small number of subgroups or “clusters,” each of which has a distinct variant algorithm.

As used herein, the process of creating one or more algorithms for the conversion of tissue fluorescence data for a person or group into blood glucose values for that same person or group will be referred to as the “fluorescence-glucose calibration problem,” or when no confusion could exist, more simply as “glucose calibration.”

In the case of in-vivo tissue auto-fluorescence spectra, it has been established that a correlation between the spectra and glucose exists. This can be seen, for instance, by comparing spectra associated with high glucose levels to those associated with low glucose levels. A statistically significant difference can be observed via, e.g., a t-test. However, although the spectra associated with very high and very low levels show a difference, there is still a considerable overlap between the two distributions. A method to quantitatively relate glucose levels to spectral characteristics cannot be obviously inferred from this correlation.

Furthermore, the relationship between fluorescence and glucose is indirect, i.e., glucose does not itself fluoresce, but causes some other change to the environment which influences the observed fluorescence spectrum. Therefore, there is no strong reason to assume that whatever relationship exists obeys, say, Beer's Law.

The attempt to tease out a quantitative relationship such as the glucose calibration problem generally falls under the rubric of exploratory data analysis. (Once such a relationship has been established, the same analytical techniques can be used to make a commercial instrument.) There is a very large and rapidly growing body of literature on this subject, some of which is discussed above. Most of the commonly-used analytical techniques, such as linear regression, multiple linear regression, and principal components analysis, look for linear relationships between what varies and the factors that are supposed to explain the variation, as the mathematics is much more tractable. A striking feature of the present invention is that the solution of the fluorescence-glucose calibration problem involves relationships which do not emerge when prior-art exploratory data analysis techniques are applied.

The present invention involves first pre-processing data, then applying exploratory data analysis techniques, then undoing the pre-processing in order to achieve glucose calibration.

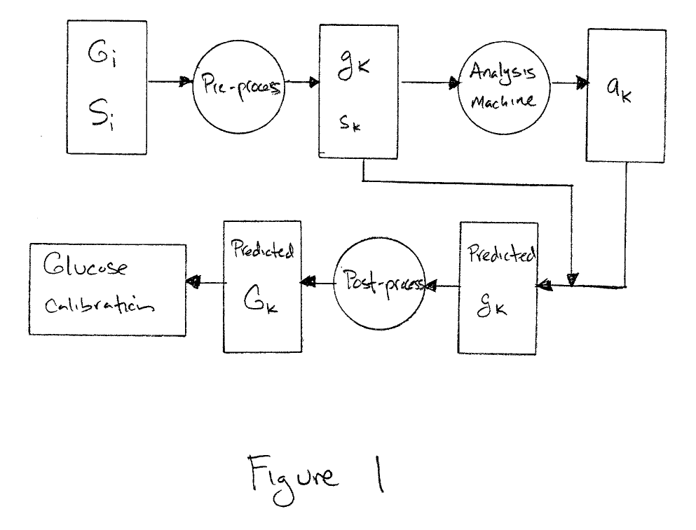

A simplified flow diagram for glucose calibration according to the invention is shown in FIG. 1. At the left of FIG. 1 are a set of glucose values Gᵢ, taken by invasive means and representing ground truth, as well as a set of ultraviolet fluorescence spectra Sᵢ taken simultaneously with the Gᵢ. These are preprocessed using algorithms which are at the core of the present invention, and converted into transformed variables (gₖ and sₖ) where the different subscript k is used to emphasize that, as part of the transformation, more than one (Gᵢ, Sᵢ) pair may be converted to a (gₖ, sₖ) pair, e.g., by averaging, as will be discussed more fully below. The transformations which have been chosen to transform (Gᵢ, Sᵢ) into (gₖ, sₖ) all express in some way the idea that the underlying relationship between fluorescence and glucose is relative, rather than absolute. That is to say, it is impossible to infer a glucose level from a single fluorescence spectrum, but given a pair of spectra (or more), it is possible to deduce the change in glucose.

Preprocessing

a. Smoothing and Averaging

Before any transformation is applied, the (Gᵢ, Sᵢ) data are typically smoothed or averaged in order to lessen the high degree of temporal and wavelength correlation that may be present. One or more of the following techniques may be employed:

(i) Banding: Two or more (Gᵢ, Sᵢ) pairs are replaced by their average, or contiguous sets of wavelengths within a given spectrum may be replaced by their average.

(ii) Smoothing: A running filter is applied to the data, so that each data point is replaced by a weighted sum of nearby points. The 5-point Chebyshev filter:

F₋₂=1/70(69f₋₂+4f₋₁−6f₀+4f₁−f₂);

F₋₁=1/35(2f₋₂+27f₋₁+12f₀−8f₁+2f₂);

F₀=1/35(−3f₋₂+12f₋₁+17f₀+12f₁−3f₂);

F₁=1/35(2f₋₂−8f₋₁+12f₀+27f₁+2f₂);

F₋₂=1/70(−f₋₂+4f₋₁−6f₀+4f₁+27f₂);

was used to smooth data in time (glucose and spectra). The same approximation was used to smooth wavelength intensities within spectra.

b. Single Point Methods

Once the smoothing and averaging has been done, the data are then transformed by either “single point” or “point-by-point” methods. In single point methods, all of either the Gₖ or the Sₖ, or both, are operated on by one single (G|S)_{N}. The notation (G|S) is used to mean “either G or S, as appropriate.” N here is used to denote some fixed member of the ensemble of glucose values and spectra. The first one was most often used, but other ones are also effective to different degrees. Single point methods are selected from the following group: (g|s)ₖ=(G|S)ₖ−(G|S)_{N} or (g|S)ₖ=(G|S)ₖ÷(G|S)_{N}.

c. Point-by-Point Methods

In point-by-point point methods, the Gₖ or the Sₖ or both are operated on by the glucose or spectrum that precedes it in the time series. Point-by-point methods are selected from the following group: (g|s)ₖ=(G|S)ₖ−(G|S)ₖ₋₁ or (g|S)ₖ=(G|S)ₖ÷(G|S)ₖ₋₁. Group members can be intermixed—that is, the transformation gₖGₖ−Gₖ₋₁ may be used in combination with sₖ=Sₖ÷Sₖ₋₁. Note particularly that the effect of transformation (g|s)ₖ=(G|S)ₖ÷(G|S)ₖ₋₁ is highly non-linear after it has been applied sequentially to elements of a time series.

Analysis

The terminology “analysis machine” in FIG. 1 is used to emphasize the fact that “standard” multivariate analysis techniques with “standard” pre-processing are used to build a statistical model relating the gₖ to the Sₖ. Pre-processing consists of mean subtraction and variance scaling, while the multivariate technique is typically Partial Least Squares, from either a commercial statistics package, such as SAS, or PLS Toolkit from the commercial mathematical software Matlab. Other techniques, such as Multiple Linear Regression and Stepwise Linear Regression can also employed with similar results. As noted above, using the same techniques to relate the Gᵢ to the Sᵢ, but without pre-processing, resulted in statistical models with significantly inferior performance.

Post-Processing

In post-processing, the statistical model relating the gₖ to the sₖ (denoted as aₖ in FIG. 1) is then combined with the transformation taking the (g|s)ₖ back to (G|S)ₖ to create a model in the original glucose and spectra space. This can then be used for various types of prediction to evaluate the model's performance (and eventually to predict glucose from spectra in a final device.)

Although the description above has used the example of glucose, those skilled in the art will immediately appreciate that the methodology may be extended to other processes with indirect effects, that is, ones in which the ultimate analyte of interest is not being directly measured, but instead through its effects on its environment.

Other embodiments and uses of the invention will be apparent to those skilled in the art from consideration of the specification and practice of the invention disclosed herein. All references cited herein, including all U.S. and foreign patents and patent applications are specifically and entirely hereby incorporated herein by reference. These include, but are not limited: to U.S. patent application Ser. No. 09/704,829, titled “Asynchronous Fluorescence Scan,” filed Nov. 3, 2000; U.S. patent application Ser. No. 09/785,550, titled “Reduction of Inter-Subject Variation Via Transfer Standardization,” filed Feb. 18, 2001; U.S. patent application Ser. No. 09/785,531, titled “Multivariate Analysis of Green to Ultraviolet Spectra of Cell and Tissue Samples,” filed Feb. 18, 2001; and U.S. patent application Ser. No. 09/785,549, titled “Generation of Spatially-Averaged Excitation-Emission Map in Heterogenous Tissue,” filed Feb. 18, 2001. It is intended that the specification and examples be considered exemplary only, with the true scope and spirit of the invention indicated by the following claims.

We claim:

1. A method of quantitating a relationship between an analyte level in in vivo tissue and auto-fluorescent spectral characteristics in said tissue, comprising:
generating a single excitation wavelength or plurality of different excitation wavelengths of green to ultraviolet light;
irradiating the tissue with said light and measuring the intensity of the stimulated emission of the sample at a minimum of two different wavelengths of lower energy than the excitation light or at a plurality of wavelengths of lower energy than the excitation light;
applying a transformation to the wavelength data;
analyzing the transformed data; and
inverting the original transformation to yield analytical results in standard units.

2. The method of claim 1 wherein the analyte is glucose and the tissue is skin.

3. The method of claim 2 wherein relative transformations of glucose and spectra are selected from the group comprising the single-point transformations (g|s)ₖ=(G|S)ₖ−(G|S)_{N} or (g|s)ₖ=(G|S)ₖ÷(G|S)_{N} and the point-by-point transformations (g|s)ₖ=(G|S)ₖ−(G|S)ₖ₋₁ or (g|s)ₖ=(G|S)ₖ÷(G|S)ₖ₋₁.

4. A method of quantitating a relationship between an analyte level in tissue and an absorption spectrum of said tissue, wherein a concentration of said analyte is not being directly measured, but rather indirectly inferred through its effect on components of said tissue, said method comprising:
irradiating the tissue with electromagnetic radiation and measuring the absorption spectrum of said electromagnetic radiation;
applying a relative transformation to the spectral data and another relative transformation to the analyte, the relative transformation in each case being selected from a group comprising either point-by-point or single-point relative transformations;
analyzing the transformed data using multivariate techniques; and
inverting the original transformation to yield analytical results in standard units.

5. The method of claim 4 wherein the electromagnetic radiation is near-ultraviolet to visible light.

6. The method of claim 4 wherein the electromagnetic radiation is visible to near-infrared light.

7. The method of claim 4 wherein the electromagnetic radiation is infrared radiation.

8. A method of quantitating a relative relationship between a set of absolute values, Gᵢ, and a set of corresponding experimental spectra, Sᵢ, wherein each respective pair (Gᵢ, Sᵢ) within the set are acquired simultaneously, comprising the steps of:
transforming two or more of said pairs according to an algorithm into one or more transformed pairs (gₖ, Sₖ);
analyzing the set of transformed pairs (gₖ, sₖ) using an analysis technique to determine a first statistical model relating gₖ to sₖ; and
inverting said first statistical model relating gₖ to sₖ according to said algorithm to create a second statistical model relating a set of experimental values Sₖ to a set of absolute values Gₖ,
wherein said second statistical model is used to predict an absolute value of an analyte from an experimental spectrum taken of said analyte.

9. The method of claim 8 wherein said algorithm comprises a single point process.

10. The method of claim 9 wherein said single point process is selected from the group consisting of: (g|s)ₖ=(G|S)ₖ−(G|S)_{N} or (g|s)ₖ=(G|S)ₖ÷(G|S)_{N}.

11. The method of claim 8 wherein said algorithm comprises a point-by-pint process.

12. The method of claim 11 wherein said point-by-point process is selected from the group consisting of: (g|s)ₖ=(G|S)ₖ−(G|S)ₖ₋₁ or (g|s)ₖ=(G|S)ₖ÷(G|S)ₖ₋₁.

13. The method of claim 8 further comprising the step of smoothing or averaging said pairs prior to transforming.

14. The method of claim 13 wherein said averaging comprises replacing two or more of said pairs with their average.

15. The method of claim 13 wherein said smoothing comprises applying a running filter so that each data point is replaced by a weighted sum of nearby points.

16. The method of claim 15 wherein said running filter is a 5-point Chebyshev filter.

17. The method of claim 8 wherein said analysis technique is a multivariate analysis technique.

18. The method of claim 17 wherein said multivariate analysis technique comprises partial least squares analysis.

19. The method of claim 8 wherein said analyte is glucose and said experimental spectrum comprises two or more wavelengths of light emitted from a sample comprising said glucose.

20. The method of claim 19 wherein said sample is stimulated by excitation light comprising one or more wavelengths in a range of green to ultraviolet light.
